(19) United States
(12) Patent Application Publication
(10) Pub. No.: US 2019/0030238 A1
(43) Pub. Date: Jan. 31, 2019

(54) MEDICINE INFUSING APPARATUS

(71) Applicant: W&G CO.,LTD., Seoul (KR)

(72) Inventor: Byoung Chic CHO, Seongnam-si (KR)

(21) Appl. No.: 15/742,483

(22) PCT Filed: May 17, 2016
(86) PCT No.: PCT/KR2016/005212

§ 371 Date: Oct. 4, 2018

(30) Foreign Application Priority Data
Jul. 8, 2015 (KR) 10-2015-0097270

(51) Int. Cl. A61M 5/14; A61M 5/142; A61M 5/168

(52) U.S. Cl. CPC A61M 5/1408; A61M 5/16813; A61M 5/16877; A61M 5/1424

(57) ABSTRACT

The present invention relates to a medicine infusing apparatus that may conveniently and safely infuse a medicine according to a usage of the medicine with a compact configuration. The medicine infusing apparatus includes a cover, a first tube located inside the cover and through which a first medicine flows, a storage part located inside the cover and in which a second medicine discharged by an external pressure is accommodated, a second tube connected to opposite sides of the storage part and through which the discharged second medicine flows, a press part located on one side of the cover to be movable and configured to apply or release a pressure to or from the storage part as the press part is moved, and a regulation part located on another side of the cover and configured to regulate movement of the press part according to an operation thereof. According to the medicine infusing apparatus according to the present invention, the apparatus may be conveniently used even only by connecting the apparatus to a medicine supply unit with a compact configuration and may promptly infuse a medicine according to the state of the patient by conveniently manipulating the apparatus. Further, a danger of the infusion of a medicine due to the usage of the medicine may be effectively prevented.

### TECHNICAL FIELD

The present invention relates to a medicine infusing apparatus, and more particularly, to a medicine infusing apparatus that may conveniently and safely infuse a medicine according to a usage of the medicine with a compact configuration.

### BACKGROUND ART

In the medical field, medicines injected or infused into the patients naturally have to be injected according to the usages of the medicines, and among the usages, an amount of the medicine, an infusion speed, and an infusion time are very important factors.

In particular, injection of a pain killer that is necessary to manage pain accompanied after a surgery of the patient is very important, adjustment of pain after the surgery is one scale for determining a result of a surgery, and generally a time period for which the pain is most severe after the surgery is 72 hours from the surgery. The pain treatment after the surgery is a problem that has to be solved in all surgeries.

Accordingly, in 1995, U.S. pain society defined pain as a fifth vital sign.

Main medicines user to adjust pain include a muscle relaxant, a sedative, and a pain killer. The most general method used to adjust pin is to intermittently inject the medicine whenever the patient appeals pain.

However, according to the intermittent injection method, pain cannot be uniformly adjusted, and as a small amount of medicine has to be frequently infused, an excessive amount of medicine is injected so that restraint of respiration, an excessive sedation, a lung/intestine problem such as nausea-vomiting, a urinary retention, or a pruritus may be caused according to the kind of the medicine and in a severe case, drug addiction may be caused.

Accordingly, a patient-controlled analgesia (PCA) using a PCA apparatus that allows the patient to infuse a drug by himself or herself instead of adjusting pain through the intermittent infusing method has been developed.

The PCA method was developed and practiced by Sechzer, and is currently being used in various areas. The PCA method employs a PCA infuser system (or a PCA pump) that is a PCA apparatus through which the patient may directly adjust injection of a medicine while continuously infusing the medicine that shows low side-effect rate and high effect.

The combination of medicines applied to the PCA, an initial loading dose, a background infusion rate (a basal rate), a demand dose (a bolus dose), a lockout interval, and a combination of used medicines are not standardized yet, and various method are used.

The initial loading does is a dose of a medicine that is injected when a PCA system is initially applied after a surgery, and a demand dose is a set dose of a medicine that is additionally injected when the patient feels pain. The lockout interval is a time interval for which injection of a medicine is interrupted until a demand dose of medicine is injected again after the demand dose of medicine is injected, and is a time period that is set to prevent infusion of an excessive amount of medicine. The background infusion rate is an amount of medicine that is injected at a specific speed regardless of the demand dose.

Although the frequency and degree of the side-effects of the pain treatment method by the PCA are lower than those of the pain treatment method by the intermittent infusion, it is important to select a medicine and determine a background infusion rate and a demand dose according to the state of the patient because it may also cause side-effects, and in particular, it is very important to manage a lockout interval after injection of a demand dose.

Accordingly, in recent years, the PCA method is carried out by installing a pump in an electronic control apparatus to control various factors of the PCA method, but it is difficult to promptly and flexibly cope with the large-scale of the apparatus and the state of the patient.

As an example, Korean Patent Application Publication No. 201-0005182 discloses an infusion apparatus used for a PCA method, but the infusion apparatus performs a control to restrain generation of bubbles that may be generated when a demand dose of medicine is infused and to prevent additional introduction of a medicine when a stored demand dose of medicine is infused and the efficient control of the factors of the PCA method is still a problem that is to be solved.

(Patent Document 1) KR 2014-0005182 A

### DISCLOSURE

### Technical Problem

Accordingly, the present invention has been made in an effort to solve the above-mentioned problems, and provides an apparatus that may conveniently and promptly infuse a medicine injected to the patient according to the usage of the medicine and may prevent a danger of side-effects due to continuous infusion by certainly securing an interruption time.

### Technical Solution

In accordance with an aspect of the present invention, there is provided a medicine infusing apparatus including a cover, a first tube located inside the cover and through which a first medicine flows, a storage part located inside the cover and in which a second medicine discharged by an external pressure is accommodated, a second tube connected to opposite sides of the storage part and through which the discharged second medicine flows, a press part located on one side of the cover to be movable and configured to apply or release a pressure to or from the storage part as the press part is moved, and a regulation part located on another side of the cover and configured to regulate movement of the press part according to an operation thereof.

The regulation part may include a locking plate located on another side of the cover to be rotatable, and a stopping pin extending from one side surface of the locking plate toward one side of the cover, the press part may include a stopping part extending from one side thereof toward one side of the cover), and the movement of the press part may be regulated while the stopping pin is coupled to or separated from the stopping part according to a rotational direction of the locking plate.

The locking plate may include a seating part located at a central portion thereof, and a locking pin located to be inserted into and extracted from the seating part.

The rotation of the locking plate may be regulated according to a location of the locking pin.

Rotation of the locking plate may be prevented if the locking pin is located inside the seating part, and rotation of the locking plate may be allowed if the locking pin is located outside the seating part.

The medicine infusing apparatus may further include a release member coupled to the regulation part such that an operation of the regulation part is performed.

The medicine infusing apparatus may further include a release member including a release boss that is inserted into a through-hole formed in the seating part, the release member may be coupled to the regulation part, and the locking pin may be located outside the seating part if the release boss passes through the through-hole to press the locking pin, and the locking pin may be located inside the seating part if the release member is released from the regulation part.

The first tube and the second tube may be located in the cover to be linearly parallel to each other.

The medicine infusing apparatus may further include a flow rate adjusting apparatus installed in one or more passages of the first tube and the second tube.

The medicine infusing apparatus may further include a check valve installed on a discharge direction of the second tube.

The medicine infusing apparatus may further include a fastening part located on one side of the cover.

### Advantageous Effects

As described above, according to the medicine infusing apparatus according to the present invention, the apparatus may be conveniently used even only by connecting the apparatus to a medicine supply unit with a compact configuration and may promptly infuse a medicine according to the state of the patient by conveniently manipulating the apparatus.

Further, a danger of the infusion of a medicine due to the usage of the medicine may be effectively prevented.

### DESCRIPTION OF THE INVENTION

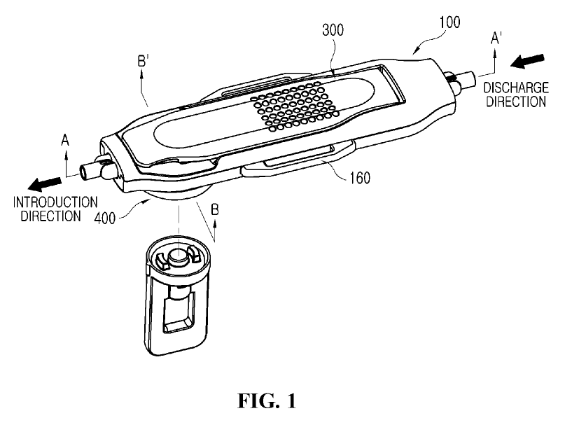

FIG. 1 is a perspective view of a medicine infusing apparatus according to an embodiment of the present invention;

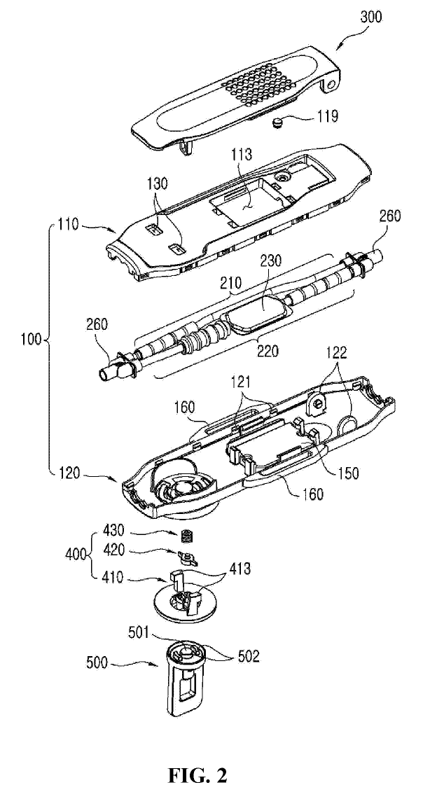

FIG. 2 is an exploded perspective view of the medicine infusing apparatus according to the embodiment of the present invention;

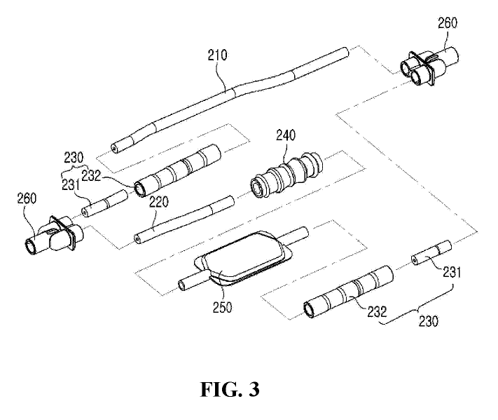

FIG. 3 is an exploded perspective view of tubes 110 and 120 and a storage part 250 that are configurations of the medicine infusing apparatus according to the embodiment of the present invention;

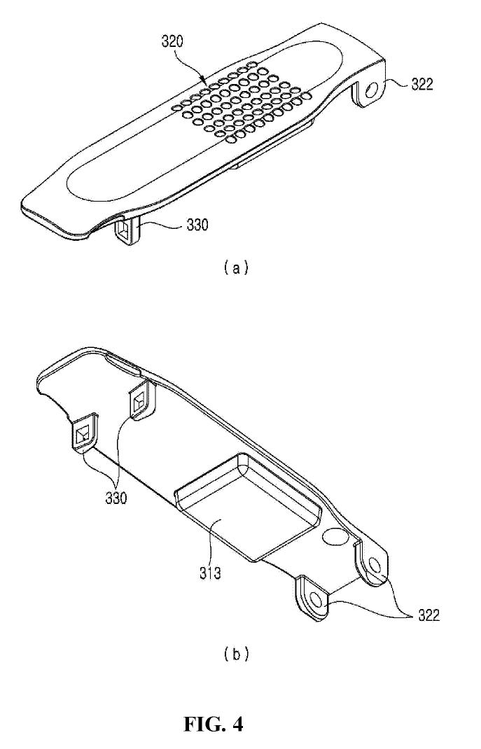

FIG. 4 illustrates perspective views of a press part that is a configuration of the medicine infusing apparatus according to the embodiment of the present invention, and illustrates perspective views viewed from the top (FIG. 4A) and the bottom (FIG. 4B);

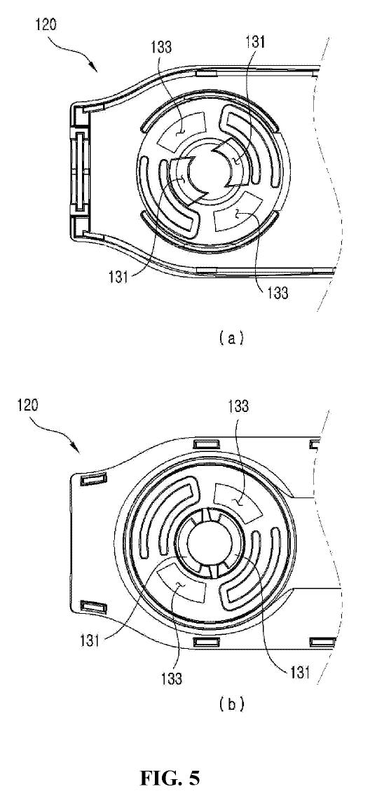

FIG. 5 illustrates a plan view and a bottom view obtained by viewing one side of a lower cover 120 that is a configuration of the medicine infusing apparatus according to the embodiment of the present invention from the top (FIG. 4A) and the bottom (FIG. 4B);

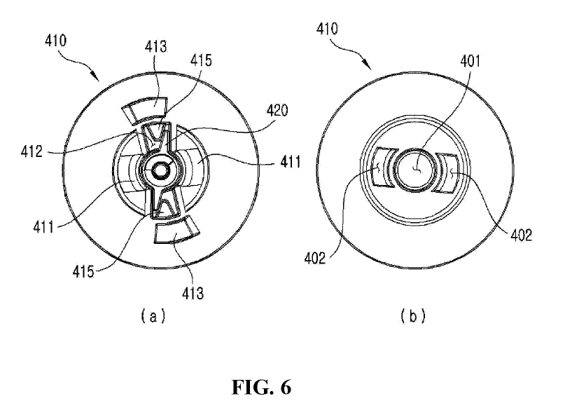

FIG. 6 illustrates a plan view and a bottom view obtained by viewing one side of a locking plate 410 of a regulation part 400 that is a configuration of the medicine infusing apparatus according to the embodiment of the present invention from the top (FIG. 4A) and the bottom (FIG. 4B);

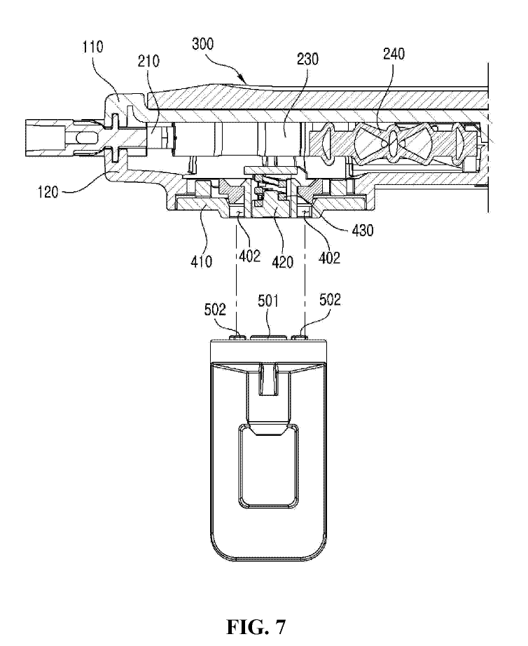

FIG. 7 is a sectional view taken along a line A-A′ of FIG. 1; and

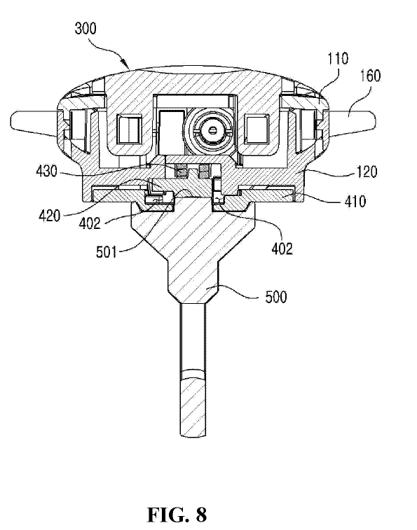

FIG. 8 is a sectional view taken along a line B-B′ of FIG. 1.

### BEST MODE

The objectives, features, and other advantages of the present invention will be more apparent by describing exemplary embodiments of the present invention in detail with reference to the accompanying drawings. It should be noted that the drawings are not to precise scale and may be exaggerated in thickness of lines or size of components for descriptive convenience and clarity. In addition, terms used herein are defined by taking functions of the present invention into account and can be changed according to user or operator custom or intention. Therefore, definition of the terms should be made according to the overall disclosure set forth herein.

Further, the embodiments are provided exemplarily for description of the present invention, and are not intended to limit the technical scope of the present invention.

The elements constituting the medicine infusing apparatus according to the present invention may be integrally used or separately used as occasion demands. Further, some elements may be omitted according to the usage of the apparatus.

Hereinafter, the medicine infusing apparatus according to an embodiment of the present invention will be described with reference to FIGS. 1 to 8.

The medicine infusing apparatus according to the embodiment of the present invention, as illustrated in FIGS. 1 and 2, may include a cover 100, first and second tubes 210 and 220, a storage part 250, a press part 300, a regulation part 400, and a release member 500.

The cover forms an external appearance of the medicine infusing apparatus, and preferably includes an upper cover 110 and a lower cover 120, a plurality of coupling recesses 111 formed along an outer peripheral surface of the upper cover 110 and a plurality of coupling bosses 121 formed along an inner peripheral surface of the lower cover 120 are coupled to each other through snap coupling to form a specific space in the interior thereof, and additionally, may be coupled by a cover fixing member 119 that passes through one side of the upper cover 110 to be connected to an inside of the lower cover 120.

The first tube 210 is located along a lengthwise direction of the cover 100 from an inside of the cover 100, and a first medicine is introduced through one side of the first tube 210 and is discharged to an opposite side.

The second tube 220 is located along a lengthwise direction of the cover 100 from an inside of the cover 100.

Opposite sides of the storage part 250 are connected to the second tube 220 to be located inside the cover 100, and preferably, are supported by a storage part support body 150 provided inside the lower cover 120 to be fixed inside the cover 100.

That is, the first tube 210 and the second tube 220 are located in parallel to each other along a lengthwise direction of the cover 100, and preferably, opposite ends of the tubes 210 and 220 are connected to branch parts of a Y-connection pipe 260 so that the tubes 210 and 220 may be disposed in parallel to each other inside the cover 100.

A specific space is provided in the interior of the storage part 250 such that a second medicine may be accommodated in the specific space, and the accommodated second medicine is discharged from the storage part 250 by a pressure applied by the storage part 250 to be discharged through the second tube 220.

The first medicine and the second medicine are not limited, and as an example, the first medicine may be a medicine that requires continuous injection and may be a pain adjusting medicine injected according to the infusion rate, and the second medicine may be a pain adjusting medicine an amount of which corresponds to a dose, which is infused once, according to a preset demand dose amount that is additionally injected when the patient feels pain.

In the medicine infusing apparatus, the storage part 250 in which the second medicine is stored is generally far away from the patient for control thereof and disposed individually so that it is often difficult to promptly cope with a situation of the patient, and the medicine infusing apparatus according to the present invention includes a first tube 210, a second tube 220, and a storage part 250 in the interior of a compact cover to be conveniently used and controlled even only by connecting the tubes 210 and 220 to a medicine supply unit (not illustrated), and preferably, a member such as a belt is fastened to a fastening part 160 located on one side of the cover 100 such that the medicine infusing apparatus may be conveniently attached to the body of the patient.

Meanwhile, as illustrated in FIG. 3, flow rage adjusting apparatuses 230 for adjusting the flow rates of the flowing medicines are provided on the passages of the tubes 210 and 220. As illustrated, the flow rate adjusting apparatus 230 includes a glass tube 231 connected to sides of the tube 210 and 220, and a support that facilitates connections of the glass tube 231 and the tube 210 and 220 and surrounds a circumference of the glass tube 231 to support the glass tube 231. By adjusting the inner diameter of the glass tube 231, the flow rates of the medicine flowing through the tubes 210 and 220 may be adjusted.

A check valve 240 is installed on one side of the storage part 250 of the second tube 220, in detail, in a discharge direction of the second medicine discharged from the storage part 250 so that the medicine introduced into the storage part 250 through an inlet side of the second tube 220 is discharged to an outlet side before it is fully filled in the storage part 250 to prevent the medicine from being injected to the patient and to allow the second medicine to be discharged from the storage part 250 and discharged through the second tube 220 only when a specific pressure is applied to the storage part 250 even after the storage part 250 is fully filled with the second medicine.

The press part 300 is located on one side of the cover to be movable, and may apply or release a pressure to the storage part 250 located inside the cover 100 through movement thereof. Any means capable of applying a pressure from one side of the cover 100 such that the second medicine accommodated in the storage part 250 may be discharged is sufficient as a configuration of the press part 300.

In detail, as illustrated in FIGS. 2 and 4, a hinge opening 322 located on one side of the upper cover 110 passes through the upper cover 110 to be coupled to a hinge member 122 located on one side of the interior of the lower cover 120 so that the upper cover 110 may be hinged about the coupling part, and a protruding press member 313 is located on a surface of the press part 300 that is close to the cover 100 so that the press member 300 may apply or release a pressure to the storage part 250 by a hinge movement of the press part 300 through a press window 113 opened on the press part 300 of the cover 100.

Additionally, a medicine display window 320 formed of a transparent material may be provided on one side of the press part 300 so that the second medicine accommodated in the storage part 250 may be identified through the press window 113.

Accordingly, the user may promptly discharge and infuse the second medicine with a simple operation of pressing the press part 300 with a hand Meanwhile, when the usage of the second medicine is particular according to the kind of the second medicine or a side effect or a danger is caused by the injection timing, the simple infusion of the second medicine may be dangerous, and the regulation part 400 is provided as a means for regulating the infusion.

The regulation part 400 is a means that is located on one side of the lower cover 120 and capable of regulating movement of the press part 300 by providing a regulation means capable of regulating movement of the press part 300 in the lower cover 120.

In detail, the regulation part 400 may include a locking plate 410, a stopping pin 413, and a locking pin 420.

As illustrated in FIGS. 2, 5, and 6, the locking plate 410 is coupled to be rotatable through the coupling member 411 located around a central axis thereof and a coupling recess 131 located on one side of the lower cover 120, preferably, through snap coupling.

A seating part 412 having a specific space is provided around the central axis of the locking plate 410, in detail, inside the coupling member 411, a locking pin 420 that is inserted into and extracted from the seating part 412 is provided, the locking pin 420 is fixed not to be rotated, and an elastic member 430 that elastically support the locking pin 420 may be located between the locking pin 420 and the lower cover 120.

Regulation bosses 415 protruding toward the inside of the seating part 412 may be located at opposite lengthwise ends of the seating part 412, and opposite ends of the locking pin 420 may be located on opposite sides of the regulation bosses 415 as the locking plate 410 is rotated. Further, a through-hole 401 is formed on a lower surface of the seating part 412.

The stopping pin 413 extends from a side surface of the lower cover 120 of the locking plate 410 toward the lower cover 120, and the stopping pin 413 is located inside the cover after passing through the through-hole 133 formed in the lower cover 120 to be moved as the locking plate 410 is rotated so that an operation of the press part 300 may be regulated by preventing or allowing a hinge movement of the press part 300 as the stopping pin 413 extends from a side surface of the upper cover 110 of the press part 300 toward the upper cover 110 to be coupled to or separated from the stopping part 330 that passed through the upper cover 110.

The release member 500 includes a release boss 501 located on one side thereof, and an insertion boss 502 located around the release boss 501.

The locking plate 410 is rotated as the release member 500 is coupled to the regulation part 400 to be rotated.

Preferably, the release boss 501 is located at a central portion of one side of the release member 500 so that the locking pin 420 is pressed to be moved to the outside of the seating part 412 as the release boss 501 is inserted into the through-hole formed in the seating part of the locking plate 410 when the regulation part 400 of the release member 500 is coupled (see FIG. 8).

When the regulation part 400 of the release member 500 is coupled, the insertion boss 502 is inserted into a recess located on an opposite side surface of the lower cover 120 of the locking plate 410 so that the locking plate 410 is rotated as the release member 500 is rotated (see FIG. 7).

Next, an operation of the medicine infusing apparatus according to the present invention will be described.

As illustrated in FIGS. 1, 7, and 8, the press part 300 is adhered to the upper cover 110, and the first medicine is continuously introduced through the first tube 210 while the movement of the press part 300 is restricted in a state in which the stopping pin 413 is coupled to the stopping part 330. Further, because the storage part 250 continues to be pressed between the press part 300 and the lower cover 120 by the press member 313 located in the press part 300, the second medicine cannot be introduced into the storage part 250 through the second tube 220 to be stored.

Further, in this state, the locking pin 420 is stopped by the regulation bosses located in the seating part to prevent rotation of the locking plate 410. That is, the press part 250 is prevented from being operated by rotating the locking plate 410 by force by using a hand or another tool.

In this state, when it is necessary to infuse the second medicine, the release boss 501 passes through the through-hole formed in the seating part by coupling the regulation part 400 to the release member 500 so that the locking pin 420 is separated to the outside of the seating part by pressing the locking pin 420 as illustrated in FIG. 8.

Accordingly, the locking plate 410 maintained by the locking pin 420 and the regulation bosses is released to be rotated, and the locking plate 410 is rotated as the insertion boss 502 inserted into the recess of the locking plate 410 is moved if the release member 500 is rotated in this state, and accordingly, the stopping pin 413 is moved to be separated from the stopping part 330 and the press part 300 may be hinged upwards and downwards.

Accordingly, the second medicine is introduced through an inlet side of the second tube 220 to be introduced into and stored in the storage part, and the discharge of the second medicine is prevented by the check valve 240 located on an outlet side of the second tube 220 as described above while the second medicine is stored.

After it is identified through the medicine display window 320 that the second medicine is fully filled in the storage part 250, a pressure is applied to the storage part 250 by pressing the press part 300 so that the second medicine accommodated in the storage part 250 exceeds a pass pressure of the check valve 240 and is discharged and infused into the patient.

As described above, an operation of the press part 300 is allowed only by the release member 500 so that the second medicine may be infused. That is, the release member 500 functions as a key.

Accordingly, because the release member 500 is managed by a medical expert (a doctor or a nurse) after the second medicine is infused, the second medicine may be injected and a lockout interval may be safely managed so that a danger due to a misuse of the second medicine may be prevented.

As described above, the medicine infusing apparatus of the present invention may be conveniently used even only by connecting the first tube and the second tube to the medicine supply unit because the first tube, the second tube, and the storage part are provided inside the compact tube.

Further, the second medicine may be promptly discharged and infused through a simple operation of pressing the press part with a hand, and a danger of side effects due to a deficiency of a usage for injection of the second medicine may be effectively prevented.

Although the preferred embodiments of the present invention have been described, the present invention is not limited to the embodiments. That is, the present invention may be variously changed and corrected by those skilled in the art to which the present invention pertains without departing from the spirit and scope of the claims, and all the modifications and equivalents have to be regarded as pertaining to the scope of the present invention.

1. A medicine infusing apparatus comprising:
a cover (100);
a first tube (110) located inside the cover (100) and through which a first medicine flows;
a storage part (250) located inside the cover (100) and in which a second medicine discharged by an external pressure is accommodated;
a second tube (120) connected to opposite sides of the storage part (250) and through which the discharged second medicine flows;
a press part (300) located on one side of the cover (100) to be movable and configured to apply or release a pressure to or from the storage part (250) as the press part (300) is moved; and
a regulation part (400) located on another side of the cover (100) and configured to regulate movement of the press part (300) according to an operation thereof.

2. The medicine infusing apparatus of claim 1, wherein the regulation part (400) includes a locking plate (410) located on another side of the cover (100) to be rotatable, and a stopping pin (413) extending from one side surface of the locking plate (410) toward one side of the cover (100),
wherein the press part (300) includes a stopping part (330) extending from one side thereof toward one side of the cover (100), and
wherein the movement of the press part (300) is regulated while the stopping pin (413) is coupled to or separated from the stopping part (330) according to a rotational direction of the locking plate (410).

3. The medicine infusing apparatus of claim 2, wherein the locking plate (410) includes a seating part (412) located at a central portion thereof, and a locking pin (420) located to be inserted into and extracted from the seating part (412).

4. The medicine infusing apparatus of claim 3, wherein the rotation of the locking plate (410) is regulated according to a location of the locking pin (420).

5. The medicine infusing apparatus of claim 3, wherein rotation of the locking plate (410) is prevented if the locking pin (420) is located inside the seating part (412), and rotation of the locking plate (410) is allowed if the locking pin (420) is located outside the seating part (412).

6. The medicine infusing apparatus of claim 1, further comprising:
a release member (500) coupled to the regulation part (400) such that an operation of the regulation part (400) is performed.

7. The medicine infusing apparatus of claim 5, further comprising:
a release member (500) including a release boss (501) that is inserted into a through-hole (401) formed in the seating part (412),
wherein the release member (500) is coupled to the regulation part (400), and the locking pin (420) is located outside the seating part (412) if the release boss (501) passes through the through-hole (401) to press the locking pin (420), and
wherein the locking pin (420) is located inside the seating part (412) if the release member (500) is released from the regulation part (400).

8. The medicine infusing apparatus of claim 1, wherein the first tube (110) and the second tube (120) are located in the cover (100) to be linearly parallel to each other.

9. The medicine infusing apparatus of claim 8, further comprising:
a flow rate adjusting apparatus (230) installed in one or more passages of the first tube (110) and the second tube (120).

10. The medicine infusing apparatus of claim 8, further comprising:
a check valve (240) installed on a discharge direction of the second tube (220).

11. The medicine infusing apparatus of claim 1, further comprising:
a fastening part (160) located on one side of the cover (100).
